(19) United States
(12) Patent Application Publication
(10) Pub. No.: US 2009/0163887 A1
(43) Pub. Date: Jun. 25, 2009

(54) Odor control cellulose granules with quinone compounds

(76) Inventors: Kelly D. Arehart, Roswell, GA (US); Franz Aschenbrenner, Kastl (DE); Annaig Gael Della Monta, Forchheim (DE)

Correspondence Address: KIMBERLY-CLARK WORLDWIDE, INC.;Catherine E. Wolf, 401 NORTH LAKE STREET, NEENAH, WI, 54956, US

(21) Appl. No.: 12/004,133

(22) Filed: Dec. 20, 2007

(51) Int. Cl. A61F 13/49; A61K 31/122; A61K 9/14; B32B 5/02

(52) U.S. Cl. 604/359; 424/76.1; 424/494; 424/489; 442/96

(57) ABSTRACT

A cellulose-based granule that functions as a carrier vehicle for an odor control agent is described. The granule has a relatively porous substrate body having a high-surface area of at least about 200 m²/gram, and a quinone compound either adhered to a surface or interfolded among the cellulose-based substrate. The odor control granule is free-flowing (i.e., do not exhibit a tendency to agglomerate to each other) and exhibit powder-like characteristics, wherein the granules have an average powder density of 30 to 600 g/l according to DIN 53,468.

### RELATED APPLICATION

The present application is related to U.S. patent application Ser. No. 10/955316, filed on Sep. 30, 2004.

### FIELD OF INVENTION

The present invention pertains to a means for delivering an odor controlling agent in measured doses for a variety of applications. In particular, the present invention involves a cellulose-based carrier that has on its surface quinone compounds, forming dispensable particles with high-surface area.

### BACKGROUND

Over the years, many attempts have been made to formulate an effective odor removal system and various consumer products are available for combating odiferous compounds. Some products are designed to cover up odors by emitting stronger, more dominant scents, such as may be found in scented air freshener sprays and candles. Another way to combat odiferous compounds, including ammonia, methyl mercaptan, trimethylamine, and other various sulfides and amines, is to remove these compounds from a medium by using deodorizing agents that diminish the presence of the odiferous compounds in the environment.

Activated charcoal and sodium bicarbonate are two compounds commonly used to absorb odors. The deodorizing ability of activated charcoal, however, varies based on the carbon source and the activation method and can have a low deodorizing ability, particularly for ammonia or when in the presence of moisture. Further, the black color of charcoal detracts from what consumer desire as aesthetically pleasing characteristics in otherwise white colored products. Sodium bicarbonate, and other white-colored odor absorbents such as silica gel and zeolites, are generally less effective deodorizers than activated charcoal and are therefore are less desirable to use.

Titanium oxide particles, such as taught in U.S. Pat. No. 5,480,636 issued to Maruo et al., are also useful in removing a few odors such as ammonia. U.S. Pat. No. 5,480,636 teaches adding zinc oxy or silicon oxy compounds to the titanium oxide to broaden the titanium oxide deodorizing capabilities. This approach, however, is limited by the photocatalytic nature of the titanium dioxide which requires light in order to convert odorous compounds into non-odorous compounds and as disclosed in U.S. Pat. No. 5,480,636, the titanium oxide compound's inability to function in aqueous solutions.

Within the odor control technology area, many have tried to improve odor control either by means of developing novel odor-absorbent compounds or through optimizing delivery of known odor control agents. A need exists for an odor removal compound and a delivery mechanism for said compound that is effective in both dry and moist environments. The delivery mechanism should be generate effective for odor removal either as a stand-alone substance that can be easily applied to various surfaces and materials or in various industrial and consumer products.

### SUMMARY OF THE INVENTION

Functionalized, porous, cellulose-based granules can serve as a novel delivery mechanism for certain odor-absorbent chemistries. Powder-like granules are made from discrete, interwoven cellulose strands that serve as a carrier vehicle or substrate for metal-modified silica particles, which maintain absorbent efficacy, as well as provide flexibility in fabrication into different kinds of products and delivery processes for various uses. The functional granules can be broadly applicable to odor control products.

The present invention relates to a functionalized cellulose-based substrate for an odor control composition that comprises an odor-reducing quinone compound and optionally high surface area particles. The odor-reducing quinone compound, according to an embodiment, is an anthraquinone having the following structure:

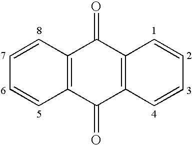

wherein the numbers 1 through 8 refer to optional substitution positions for functional groups. For example, positions 5 through 8 of the anthraquinone may be unsubstituted with functional groups. Examples of such quinone compounds may include those obtained from a dye selected from the group consisting of Acid Blue 25, Acid Green 41, Acid Blue 45, Mordant Violet 5, Acid Blue 129, Acid Green 25, D&C Green No. 5, Acid Green 27, and combinations thereof.

The cellulose-based substrate can be employed as a free-standing odor absorbent in a powder form. Alternatively, since the cellulose-based granules tend to be free-flowing, even when wet from exposure to a liquid, the granules can be loaded in self-contained pouches that allow air-flow or sprinkled loosely in packaging, for use as a general absorbent, without the mess or other problems associated with conventional ink or liquid-based delivery vehicles. The granules can be incorporated into various industrial and consumer products including absorbent articles, air and water filters, household cleaners, fabrics, and paper towels.

The invention, in another aspect, relates to an article of manufacture, such absorbent articles that are used for personal care products. The odor-control carrier substrate can be incorporated via a dosed delivery, similar to current industry methods employed to dosing superabsorbent materials into diapers or feminine hygiene products, an adult incontinence product, a protective garment, or used directly in a raw particle form for household applications like carpet or garbage odor control. The fluffy, granule nature of the carrier vehicle permits one to provide a predetermined or specified amount of odor control material into the products. The cellulose-based granule particles can be broadly applicable to odor control products, an air-freshener medium or package, air-filter medium. The medium can have the cellulose-based carrier substrates located in interstitial spaces of a nonwoven fabric web. A manufacturer can deposit a precise amount of activated odor control material accurately on locations of a substrate or incorporated the material within like products.

### BRIEF DESCRIPTION OF FIGURES

FIG. 1, is a graph showing the plot of average % removed (♦) and average amount (mg/g) removed (▪) vs. the odorant granule samples containing control technologies exposed to triethylamine (3.0 μl).

FIG. 2, is a graph showing the plot of average % removed (♦) and average mg/g removed (▪) vs. the various odorant granule samples containing odor control technologies exposed to ethyl mercaptan (2.4

### DETAILED DESCRIPTION OF THE INVENTION

### Section 1.—Definitions

Before describing the present invention in detail, the present invention is not necessarily limited to specific compositions, reagents, process steps, or equipment, as such may vary. As used in this specification and the appended claims, the singular forms “a,” “an,” and “the” include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

As used herein, the term “zeta potential” refers to the potential gradient that arises across an interface. Zeta potential measurements may be taken using, for instance, a Zetapals instrument available from the Brookhaven Instrument Corporation of Holtsville, N.Y. For example, zeta potential measurements may be conducted by adding one to three drops of a sample into a cuvet containing 1 millimolar KCl solution, using the instrument's default functions preset for aqueous solutions.

As used herein, an “absorbent article” refers to any article capable of absorbing water or other fluids. Examples of some absorbent articles include, but are not limited to, personal care absorbent articles, such as diapers, training pants, absorbent underpants, adult incontinence products, feminine hygiene products (e.g., sanitary napkins), swim wear, baby wipes, and so forth; medical absorbent articles, such as garments, fenestration materials, underpads, bandages, absorbent drapes, and medical wipes; food service wipers; textile fabrics; clothing articles; and so forth. Materials and processes suitable for forming such absorbent articles are well known to those skilled in the art.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, bonded carded web processes, etc.

As used herein, the term “meltblowing” refers to a process in which fibers are formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin et al., which is incorporated herein in its entirety by reference thereto for all purposes. Generally speaking, meltblown fibers may be microfibers that may be continuous or discontinuous, are generally smaller than 10 microns in diameter, and are generally tacky when deposited onto a collecting surface.

As used herein, the term “spunbonding” refers to a process in which small diameter substantially continuous fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel et al., U.S. Pat. No. 3,692,618 to Dorschner et al., U.S. Pat. No. 3,802,817 to Matsuki et al., U.S. Pat. No. 3,338,992 to Kinney, 3,341,394 to Kinney, U.S. Pat. No. 3,502,763 to Hartman, U.S. Pat. No. 3,502,538 to Levy, U.S. Pat. No. 3,542,615 to Dobo et al., and U.S. Pat. No. 5,382,400 to Pike et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns.

### Section II.—Description

The present invention relates to an odor-control delivery mechanism that can be employed independently by itself or as part of a product or article. The present invention builds upon the work done by MacDonald et al., such as described in U.S. Patent Publication No. 2005/0131363, the contents of which are incorporated herein by reference. Quinone compounds are combined with a substrate that has a physical form and texture of fine cellulose-based granules, such as described in International Patent Application No. WO 2006/048280, by Ozersky et al., which describes multi-functional surface-modified cellulose-containing fibers for use in making paper, tissue and cardboard products, and a process for manufacturing the granules. The contents of WO 2006/048280 are incorporated herein by reference. The granules afford manufacturers and. consumers a novel and easily quantifiable or measurable a carrier vehicle for odor control applications.

The cellulose fibers according to the present invention are at least partially in the form of granules may be completely in the form of granules, essentially in the form of granules or a mixture of fibers and granules. Both the granules and the mixture are free-flowing, which is associated with the advantages described above. The granules can be described as “saw-dust-like” or have a consistency like that of confectionary sugar. The granules tend to be irregularly shaped and have an average gross dimension in the range of about 20-3000 micrometers (μm). According to a preferred embodiment of the present invention, the particles in the granules have a diameter of 150 to 2500 μm. Typically, the granules range between about 200 or 700 μm to about 1200 μm or 1500 μm. It is especially preferable for them to have a diameter of 400-500 μm. The granules are constituted with a porous cellulose-based body or substrate having a high-surface area of at least about 200 m²/gram of material. An average powder density of the granules (measured according to DIN 53,468) of 30 to 600 g/l is also preferred. An average powder density of 100-300 g/l.±0.15% is especially preferred here. For example, the granules may be granules of the product Vitacel LC 200, cellulose fibers from the company J. Rettenmaier & Sohne GmbH Co. [J. Retternaier and Sons Inc.], Germany.

Various kinds of natural fibers may be used in the making of the granules, such as wood pulp fibers, corn silk, hemp, flax or cotton fibers, or other plant cellulose. Cellulose fibers obtained from wood and at least partially in the form of granules are preferred. Other suitable plant fibers comprise, for example, apple fibers, orange fibers and wheat fibers. Those skilled in the art will be familiar with methods of producing cellulose fibers from wood and/or other plant fibers so that they are at least partially in the form of granules. Quinone compounds are either adhered to, interfolded among, or encapsulated within or by the cellulose-based carrier substrate, such that each of the quinone compounds can bind a molecule of a gaseous compound, an odorous compound, or combinations thereof. Hence, the granules function as a carrier vehicle for an odor control agent.

The cellulose-based granules have a structure suitable for suppressing odors. The cellulose fibers according to the present invention have advantages with regard to the amount of absorbent material to be used. In comparison with polyacrylates which are used as superabsorbers, the cost of the granules is approximately of the same order of magnitude as the cost of conventional cellulose.

In certain embodiments, the cellulose fibers of this invention can bee at least partially in the form of granules; these fibers will preferably have an average fiber length of about 100 μm to about 600 μm. An average fiber length of about 300 μm is especially preferred. According to a preferred embodiment of the present invention, the average fiber thickness of the fibers is about 10 μm to about 50 μm. An average fiber thickness of 20 μm is particularly desirable.

If the cellulose fibers, at a portion of which is in the form of granules, are in the form of a mixture of fibers and granules, this may be a “true” mixture, i.e., the cellulose fibers and the resulting granules are brought in contact and mixed by methods with which those skilled in the art are familiar. On the other hand, the mixture may also consist of particles which are in the form of granules, but fibers are present on the surface of the individual granule particles. Such “mixtures” can be produced by suppressing the process of winding fine cellulose fibers or granulating them to granules so promptly that individual unwound fibers or incompletely granulated fibers project out of the granules. The mixture of granules and fibers preferably includes from 10 wt % to 80 wt % fibers. It is especially preferred for the mixture to contain 60-70 wt % granules and 30 to 40 wt % fibers. In addition to the cellulose fibers the granules may also contain other materials, such as synthetic polymer components, such as: superabsorbers, superabsorbent materials in particle form, superabsorbent fibers, viscose staple fiber or synthetic staple beads of different lengths, polyolefins, such as polyethylene, or polystyrene, etc.

As stated above, the present invention provides a delivery vehicle or carrier mechanism by which an odor control composition that includes a quinone compound, such as developed by MacDonald et al., can be effectively adhered to a surface, interfolded among, or encapsulated by a cellulose-based granule. Generally, quinones refer to a class of compounds that possess a quinoid ring, such as anthraquinones, naphthaquinones, benzoquinones, hydroquinones, and so forth. Anthraquinones, for instance, have the following general formula:

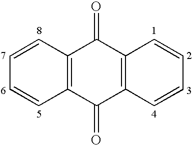

The numbers shown in the general formula represent a location on the fused ring structure at which substitution of a functional group may occur. Some examples of such functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, hydroxy groups, phosphorous groups, etc. Functional groups that result in an ionizing capability are often referred to as “chromophores.” Substitution of the ring structure with a chromophore causes a shift in the absorbance wavelength of the compound. Thus, depending on the type of chromophore (e.g., hydroxyl, carboxyl, amino, etc.) and the extent of substitution, a wide variety of quinones may be formed with varying colors and intensities. Other functional groups, such as sulfonic acids, may also be used to render certain types of compounds (e.g., higher molecular weight anthraquinones) water-soluble.

Anthraquinone compounds may be classified for identification by their Color Index (CI) number, which is sometimes called a “standard.” For instance, some suitable anthraquinones that may be used in the present invention, as classified by their “CI” number, include Acid Black 48, Acid Blue 25 (D&C Green No. 5), Acid Blue 40, Acid Blue 41, Acid Blue 45, Acid Blue 129, Acid Green 25, Acid Green 27, Acid Green 41, Mordant Red 11 (Alizarin), Mordant Black 13 (Alizarin Blue Black B), Mordant Red 3 (Alizarin Red S), Mordant Violet 5 (Alizarin Violet 3R), Natural Red 4 (Carminic Acid), Disperse Blue 1, Disperse Blue 3, Disperse Blue 14, Natural Red 16 (Purpurin), Natural Red 8, Reactive Blue 2, and so forth. For instance, the structures of Acid Blue 25, Acid Green 41, Acid Blue 45, Mordant Violet 5, Acid Blue 129, Acid Green 25, and Acid Green 27 are set forth below:

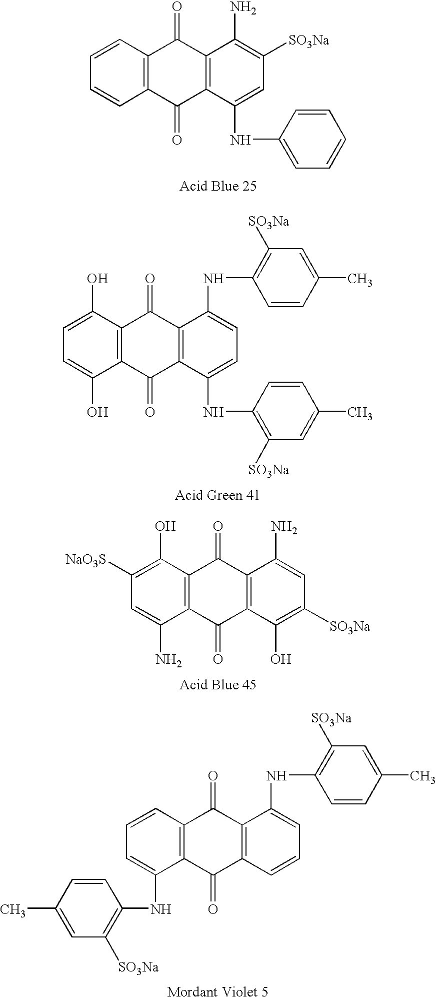

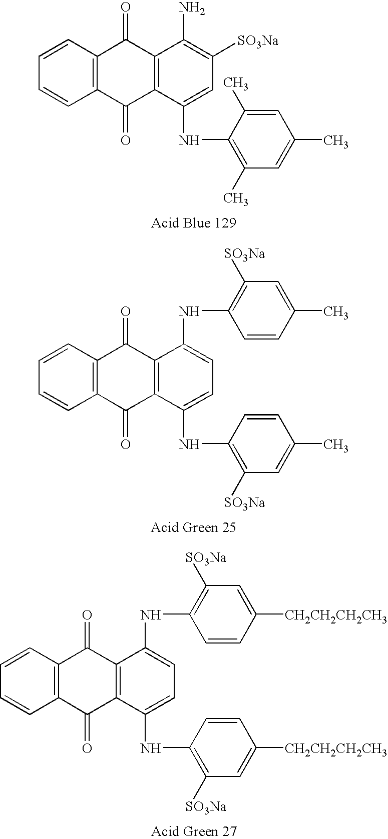

Besides their well-known ability to impart color, the present inventors have unexpectedly discovered that certain quinone compounds may also eliminate odor. Without intending to be limited by theory, it is believed that the odor caused by many compounds is eliminated by the transfer of electrons to and/or from the odorous compound. Specifically, electron reduction of odorous compounds via a reduction/oxidation (“redox”) reaction is believed to inhibit the production of the characteristic odor associated therewith. The surprising discovery that certain quinone compounds are able to eliminate odor is believed to be due their ability to function as an oxidizing agent in a redox reaction. Many common odorous compounds are capable of oxidizing (i.e., donate electrons) via a redox reaction. For instance, odorous compounds may include mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. Upon oxidation, the odors associated with such compounds are often eliminated or at least lessened. It is also believed that the reduction of the quinone compound via the redox reaction is readily reversible, and thus the reduced quinone compound may be re-oxidized by any known oxidizing agent (e.g., oxygen, air, etc.). The reduction/oxidation reactions are rapid and may take place at room temperature. Thus, although the odor control mechanism may consume the quinone compounds, they may simply be regenerated by exposure to air. Thus, long-term odor control may be achieved without significantly affecting the ability of the quinone compound to impart the desired color.

The ability of quinone compounds to accept electrons from another substance (i.e., be reduced) may be quantified using a technique known as redox potentiometry. Redox potentiometry is a technique that measures (in volts) the affinity of a substance for electrons—its electronegativity—compared with hydrogen (which is set at 0). Substances more strongly electronegative than (i.e., capable of oxidizing) hydrogen have positive redox potentials. Substances less electronegative than (i.e., capable of reducing) hydrogen have negative redox potentials. The greater the difference between the redox potentials of two substances (DELTA.E), the greater the vigor with which electrons will flow spontaneously from the less positive to the more positive (more electronegative) substance. As is well known in the art, redox potential may be measured using any of a variety of commercially available meters, such as an Oxidation Reduction Potential (ORP) tester commercially available from Hanna Instruments, Inc. of Woonsocket, R.I. The redox potential of the quinone compounds may, for instance, be less than about −50 millivolts (mV), in some embodiments less than about −150 mV, in some embodiments less than about −300 mV, and in some embodiments, less than about −500 mV. Although not always the case, the redox potential may vary based on the number and location of functional groups, such as sulfonic acid, on the quinone structure. For example, 2-sulfonic acid anthraquinone has a redox potential of −380 mV; 2,6-disulfonic acid anthraquinone has a redox potential of −325 mV; and 2,7-disulfonic acid anthraquinone has a redox potential of −313 mV. Likewise, 2-sulfonic acid naphthaquinone has a redox potential of −60 mV. The use of other functional groups may also have an affect on the ultimate redox potential of the compound. For example, Acid Blue 25, which also contains amino- and aramid functional groups, has a redox potential of −605 mV.

In addition to their ability to oxidize odorous compounds, the present inventors have also discovered that the chemical structure of certain quinone compounds results in improved odor elimination. For example, anthraquinone compounds that have at least one unsubstituted ring may result in better odor inhibition than those that are substituted at each ring with a functional group. Interestingly, anthraquinone compounds that are unsubstituted at the “first” ring (i.e., positions 5 through 8) appear to be particularly effective in reducing odor. Suitable examples of anthraquinone compounds that are unsubstituted at locations 5 through 8 include, but are not limited to, Acid Blue 25, Acid Blue 129, Acid Green 25, and Acid Green 27, the structures of which are set forth above.

Although the quinone compounds of the present invention are capable of achieving high levels of odor reduction, it is sometimes desired to further enhance the level of odor reduction through the use of high-surface area particles that act as a carrier for the compound. In some cases, the quinone compound is believed to form a coordinate bond with an atom of the particles (e.g., aluminum) via oxygen atoms present in the quinone structure. As used herein, a “coordinate bond” refers to a shared pair of electrons between two atoms, wherein one atom supplies both electrons to the pair. When utilized, the high surface area of such particles may provide a further method of reducing odor.

The high-surface area particles may be formed from a variety of materials, including, but not limited to, silica, alumina, zirconia, magnesium oxide, titanium dioxide, iron oxide, zinc oxide, copper oxide, organic compounds such as polystyrene, and combinations thereof. The particles may have a surface area of from about 50 square meters per gram (m.sup.2/g) to about 1000 m.sup.2/g, in some embodiments from about 100 m.sup.2/g to about 600 m.sup.2/g, and in some embodiments, from about 180 m.sup.2/g to about 240 m.sup.2/g. Surface area may be determined by the physical gas adsorption (B.E.T.) method of Bruanauer, Emmet, and Teller, Journal of American Chemical Society, Vol. 60, 1938, p. 309, with nitrogen as the adsorption gas.

The particles may possess various forms, shapes, and sizes depending upon the desired result. For instance, the particles may be in the shape of a sphere, crystal, rod, disk, tube, string, etc. The average size of the particles is generally less than about 500 microns, in some embodiments less than about 100 microns, in some embodiments less than about 100 nanometers, in some embodiments from about 1 to about 50 nanometers, in some embodiments from about 2 to about 50 nanometers, and in some embodiments, from about 4 to about 20 nanometers. As used herein, the average size of a particle refers to its average length, width, height, and/or diameter. If desired, the particles may also be relatively nonporous or solid. That is, the particles may have a pore volume that is less than about 0.5 milliliters per gram (ml/g), in some embodiments less than about 0.4 milliliters per gram, in some embodiments less than about 0.3 ml/g, and in some embodiments, from about 0.2 ml/g to about 0.3 ml/g. Without intending to be limited by theory, it is believed that particles having such a small size and high surface area may improve the adsorption capability for many odorous compounds. Moreover, it is believed that the solid nature, i.e., low pore volume, of the particles may enhance the uniformity and stability of the particles, without sacrificing their odor adsorption characteristics.

Regardless of the material used to form the high-surface area particles, the particles may be selected to possess a “zeta potential” that is opposite to a substrate to which it is applied. Although not required, the use of particles having an opposite zeta potential to the substrate may facilitate the binding of the particles thereto through ionic interaction. For example, in some embodiments of the present invention, the particles may have a positive zeta potential of greater than about +20 millivolts (mV), in some embodiments greater than about +30 mV, and in some embodiments, greater than about +40 mV. By possessing a positive surface charge, the particles are well suited for binding to a substrate that carries a negative surface charge (e.g., substrate containing cellulosic fibers) through ionic attraction. Depending upon the difference in charge between the particles and the substrate, the bond may sometimes be relatively permanent and substantive. Consequently, chemical binders or other attachment mechanisms may not be required. In some cases, the charge of the particles may also allow bonding to occur with the quinone dye through ionic attraction. For example, positively-charged particles may bond to some extent to negatively-charged quinone compounds (e.g., acid dyes).

A positive zeta potential may be imparted to the high-surface area particles of the present invention in a variety of different ways. In one embodiment, the particles are formed entirely from a positively charged material. For example, alumina particles may be used for odor reduction in accordance with the present invention. Some suitable alumina particles are described in U.S. Pat. No. 5,407,600 to Ando, et al., which is incorporated herein in its entirety by reference thereto for all purposes. Further, examples of commercially available alumina particles include, for instance, Aluminasol 100, Aluminasol 200, and Aluminasol 520, which are available from Nissan Chemical Industries Ltd. Alternatively, the positive zeta potential may be imparted by a continuous or discontinuous coating present on the surface of a core material. In some instances, these particles may actually possess a better stability over various pH ranges than particles formed entirely from positively charged materials. In one particular embodiment, for example, the particles are formed from silica particles coated with alumina. A commercially available example of such alumina-coated silica particles is Snowtex-AK, which is available from Nissan Chemical of Houston, Tex.

The nature of the odor control composition may vary depending on its intended use. For example, in some embodiments, the odor control composition may be a water-dispersible powder. In this manner, the powder uniformly disperses in the fluid so that higher concentrations of the odor-reducing compounds are placed in close proximity to the odorous compound. The presence of the powder also has an ancillary benefit of changing the color of the odorous compound, which may be more aesthetically pleasing to the consumer. In one embodiment, for instance, the odor control composition is a water-dispersible anthraquinone powder on a cellulose granule carrier, such as a powder of Acid Blue 25, Acid Green 41, Acid Blue 45, Mordant Violet 5, Acid Blue 129, Acid Green 25, or Acid Green 27. Such powders are commercially available from Sigma-Aldrich Chemical Co. of St. Louis, Mo. Other suitable anthraquinone powders, such as D&C Green No. 5, are commercially available from Noveon Hilton Davis, Inc. of Cincinnati, Ohio. If desired, the powder may be applied to a substrate (e.g., layer of an absorbent article). The cellulose granule carrier may be granules of the product Vitacel LC 200, cellulose fibers from the company J. Rettenmaier & Sohne GmbH Co. [J. Rettenmaier and Sons Inc.], Germany.

The cellulose-based granules infused with the odor control composition may also be used in bedpans, nursing homes, etc. For example, an odor control powder may be used as a stand-alone product that is dispersible in urine to reduce its odor. In another embodiment, the odor control composition may be used on walls, wallpaper, glass, toilets, and/or countertops. For instance, the odor control composition may be used in a restroom facility. Other uses include, without limitation, refrigerator mats and fabric softener sheets. The odor control composition may also be used in water treatment systems for removing compounds from well water or in toilet tanks to reduce the odors resulting from urine. The odor control composition may also be used in liquid detergents and household cleaners to remove odors. In another embodiment, the odor control composition is used as aerosol odor neutralizers/deodorants. The odor control composition is packaged with a propellant that allows spraying the odor control composition into the air for removal of gases and odorous compounds. The odor control composition may be used in a household air freshener or be used in combination with a mist emitted from a vaporizer or humidifier.

According to another embodiment of the invention, one may incorporate the cellulose-based granules infused with quinone compound in an absorbent article, such as described in U.S. Patent Application Publication No. 2006/0229580 A1, by Raidel et al., the content of which is incorporated herein by reference. As used herein, the term “absorbent article” refers to products or articles suitable for absorption, in particular for absorption of body fluids. This includes in particular the absorption of urine, blood and fecal matter. Absorbent articles, according to the present invention, are often disposable articles, but they need not necessarily be disposable. Examples of absorbent articles according to the present invention include sanitary pads, in particular sanitary napkins and panty liners, diapers, incontinence pads, bandages and similar articles. In addition, in some instances, synthetic components can also be incorporated as part of the cellulose-based granules. Some suitable synthetic fibers can include, but are not limited to, rayon fibers, ethylene vinyl alcohol copolymer fibers, polyolefin fibers, polyesters, and so forth.

An example of a technique for applying or dosing the present cellulose-based granules as a delivery mechanism in personal care products or other absorbent articles is described in detail in U.S. Patent Application Publication No. 2007/0100304 A1, by Fell et al., the content of which is incorporated herein by reference. Fell et al. describe a method for incorporating odor control agent particles into an absorbent article, in which odor control particles are “homogenously” distributed (e.g., in a substantially uniform manner) within an air-formed pulp fiber matrix of an absorbent core of an absorbent article. An absorbent core containing such a homogeneously distributed odor control particles may possess a greater surface area for contacting malodorous compounds, thereby increasing the likelihood of odor reduction.

Any of a variety of different products may be incorporated with the odor control composition in accordance with the present invention. For instance, nonwoven fabrics, woven fabrics, knit fabrics, wet-strength paper, film, foams, etc., may be applied with the odor control composition. When utilized, the nonwoven fabrics may include, but are not limited to, spunbonded webs (apertured or non-apertured), meltblown webs, bonded carded webs, air-laid webs, coform webs, hydraulically entangled webs, and so forth. In some embodiments, for example, the odor control composition may be utilized in a paper product containing one or more paper webs, such as facial tissue, bath tissue, paper towels, napkins, and so forth. The paper product may be single-ply in which the web forming the product includes a single layer or is stratified (i.e., has multiple layers), or multi-ply, in which the webs forming the product may themselves be either single or multi-layered. Normally, the basis weight of such a paper product is less than about 120 grams per square meter (gsm), in some embodiments less than about 80 gsm, in some embodiments less than about 60 grams per square meter, and in some embodiments, from about 10 to about 60 gsm.

According to another aspect, the cellulose-based granules can be dosed into a portion of an absorbent article. In one embodiment, for instance, the absorbent article includes a liquid-transmissive bodyside liner, a liquid-transmissive surge layer below the bodyside liner, a liquid-absorbent core below the surge layer, and a moisture vapor permeable, liquid impermeable outer cover below the absorbent core. A substrate treated with the odor control composition of the present invention may be employed as any one or more of the liquid transmissive (non-retentive) and absorbent layers. An absorbent core of the absorbent article, for instance, may be formed from an absorbent nonwoven web that includes a matrix of hydrophilic fibers. In one embodiment, the absorbent web may contain a matrix of cellulosic fluff fibers. One type of fluff that may be used in the present invention is identified with the trade designation CR1654, available from U.S. Alliance, Childersburg, Ala., U.S.A., and is a bleached, highly absorbent sulfate wood pulp containing primarily soft wood fibers. In another embodiment, the absorbent nonwoven web may contain a hydroentangled web. Hydroentangling processes and hydroentangled composite webs containing various combinations of different fibers are known in the art. A typical hydroentangling process utilizes high pressure jet streams of water to entangle fibers and/or filaments to form a highly entangled consolidated fibrous structure, e.g., a nonwoven fabric. Hydroentangled nonwoven fabrics of staple length fibers and continuous filaments are disclosed, for example, in U.S. Pat. No. 3,494,821 to Evans and U.S. Pat. No. 4,144,370 to Boulton, which are incorporated herein in their entirety by reference thereto for all purposes. Hydroentangled composite nonwoven fabrics of a continuous filament nonwoven web and a pulp layer are disclosed, for example, in U.S. Pat. No. 5,284,703 to Everhart, et al. and U.S. Pat. No. 6,315,864 to Anderson, et al., which are incorporated herein in their entirety by reference thereto for all purposes.

Another type of suitable absorbent nonwoven web is a coform material, which is typically a blend of cellulose fibers and meltblown fibers. The term “coform” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and so forth. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

If desired, the absorbent nonwoven web may also contain a superabsorbent material. Superabsorbents have the ability to absorb a great amount of fluid in relation to their own weight. Typical superabsorbents used in sanitary napkins may absorb anywhere from about 5 to about 60 times their weight in blood. Superabsorbent materials are produced in a wide variety of forms including, but not limited to, particles, fibers and flakes. Superabsorbents having a high mechanical stability in the swollen state, an ability to rapidly absorb fluid, and those having a strong liquid binding capacity, typically perform well in absorbent articles. Hydroxy functional polymers have been found to be good superabsorbents for this application. For example, a hydrogel-forming polymer, such as a partially neutralized cross-linked copolymer of polyacrylic acid and polyvinyl alcohol, may be utilized. After the polymer is formed, it is mixed with about a 1% anhydrous citric acid powder. The citric acid has been found to increase the ability of the superabsorbent to absorb menses and blood. This is particularly beneficial for use in a sanitary napkin or other feminine pads. The finely ground, anhydrous citric acid powder, which is void of water, along with trace amounts of fumed silica, is mixed with the polymer that may have been screened to an appropriate particle size. This mixture may also be formed into a composite or a laminate structure. Such superabsorbents may be obtained from Dow Chemical and Stockhausen, Inc., among others. This superabsorbent is a partially neutralized salt of cross-linked copolymer of polyacrylic acid and polyvinyl alcohol having an absorbency under load value above about 25. Some suitable superabsorbents are described in U.S. Pat. No. 4,798,603 to Meyers. et al., Re. 32,649 to Brandt. et al. and 4,467,012 to Pedersen, et al., U.S. Pat. Nos. 4,604,313 and 4,655,757 to McFarland, et al., U.S. Pat. No. 6,387,495 to Reeves, et al., as well as in published European Patent Application 0,339,461 to Kellenberger.

As indicated above, the odor control composition may also be incorporated into a liquid transmissive layer of the absorbent article, such as the bodyside liner or surge layer. Such liquid transmissive layers are typically intended to transmit liquid quickly, and thus generally do not retain or absorb significant quantities of aqueous liquid. Materials that transmit liquid in such a manner include, but are not limited to, thermoplastic spunbonded webs, meltblown webs, bonded carded webs, air laid webs, and so forth. A wide variety of thermoplastic materials may be used to construct these non-retentive nonwoven webs, including without limitation polyamides, polyesters, polyolefins, copolymers of ethylene and propylene, copolymers of ethylene or propylene with a C.sub.4-C.sub.20 alpha-olefin, terpolymers of ethylene with propylene and a C.sub.4-C.sub.20 alpha-olefin, ethylene vinyl acetate copolymers, propylene vinyl acetate copolymers, styrene-poly(ethylene-alp-ha-olefin) elastomers, polyurethanes, A-B block copolymers where A is formed of poly(vinyl arene) moieties such as polystyrene and B is an elastomeric midblock such as a conjugated diene or lower alkene, polyethers, polyether esters, polyacrylates, ethylene alkyl acrylates, polyisobutylene, poly-1-butene, copolymers of poly-1-butene including ethylene-1-butene copolymers, polybutadiene, isobutylene-isoprene copolymers, and combinations of any of the foregoing.

The effectiveness of the odor control composition of the present invention may be measured in a variety of ways. For example, the percent of an odorous compound adsorbed by the odor control composition may be determined using the headspace gas chromatography test as set forth herein. In some embodiments, for instance, the odor control composition containing quinone dyes is capable of adsorbing at least about 25%, in some embodiments at least about 45%, and in some embodiments, at least about 65% of a particular odorous compound. The effectiveness of the odor control composition in removing odors may also be measured in terms of “Relative Adsorption Efficiency”, which is also determined using headspace gas chromatography and measured in terms of milligrams of odor adsorbed per gram of the odor control composition. It should be recognized that the surface chemistry of any one type of odor control composition may not be suitable to reduce all types of odors, and that low adsorption of one or more odorous compounds may be compensated by good adsorption of other odorous compounds.

### SECTION III.—EXAMPLES

The following examples illustrate the functionality of the carrier vehicle according to the present invention. Granular cellulose particles prepared by J. Rettenmaier & Sons are physically blended to incorporated quinone-based compound odor control chemistries. All the granules were based on Vitacel LC 200 (fiber size of 300 microns, 75-95 g/l bulk density). As summarized in Table 1, the particles include anthraquinone dyes acid green 25 and remazol brilliant blue R, which have been proven to reduce malodors in the ORP setting.

TABLE 1
Details of cellulose granules or particles according to the present
invention with anthraquinone dye odor control chemistries.

| | |
|---|---|
| Green 5% | 5% Acid Green 25 |
| Green 10% | 10% Acid Green 25 |
| Blue 5% | 5% Remazol Brilliant Blue R |
| Blue 10% | 10% Remazol Brilliant Blue R |

Cellulose samples were weighed and placed in gas chromatograph (GC) headspace vials. Ethyl mercaptan (EtSH, 2.4 μl, 2.0 mg) or triethylamine (TEA, 3.0 μl, 2.2 mg) was added to a vial, the vial was covered with a cap and crimped shut. This was repeated for each vial. Three odorant control samples were run among with the test samples to create a reference set. Three reference samples of Vitacel LC 200, Acid green 25 and Remazol Brilliant Blue R were also included to measure the amount of odor absorbed by the substrate and pure compounds themselves. The GC was equipped with a headspace analyzer, a DB-624 column, and an FID detector (temp=250° C.). Samples were equilibrated for 10 minutes at 37° C. in the headspace analyzer prior to injection into an 85° C. loop. Injection onto the 30° C. column (EtSH) or 100° C. column (TEA) occurred at 105° C. with a run time of 5 minutes.

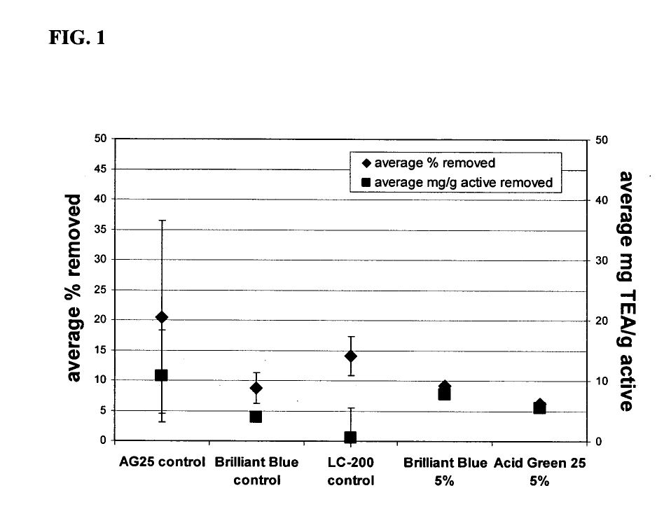

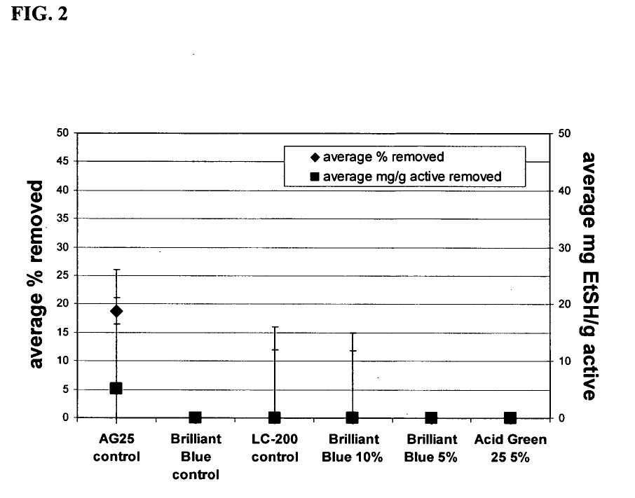

FIG. 1 details the GC headspace results obtained for the particles exposed to TEA. FIG. 2 contains the results when the particles are exposed to EtSH. For both species, there were no obvious differences in odor removal ability of the Acid green 25 or Remazol brilliant blue R when delivered on granules as compared to the pure compounds, nor an increase as compared to the LC 200 control. These results indicate the nature of the dyes remains unchanged after being subjected to the cellulose agglomeration process. It is believed that the chemical functionality or activity of the active agent is by means of a redox process, not an absorption process.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

We claim:

1. A delivery mechanism for reducing odors, the mechanism comprises a cellulose-based granule or particle substrate with an odor control agent with a composition including a quinone compound either adhered to a surface or interfolded among said cellulose-based granule.

2. The delivery mechanism of claim 1, wherein said quinone compound is an anthraquinone having the following structure: 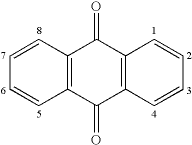
wherein the numbers 1 through 8 refer to optional substitution positions for functional groups.

3. The delivery mechanism of claim 2, wherein said anthraquinone is substituted with halogen groups, alkyl groups, benzyl groups, amino groups, carboxy groups, cyano groups, hydroxy groups, phosphorous groups, sulfonic acid groups, or combinations thereof.

4. The delivery mechanism of claim 2, wherein at least one ring of said anthraquinone is unsubstituted with functional groups.

5. The delivery mechanism of claim 4, wherein positions 5 through 8 of said anthraquinone are unsubstituted with functional groups.

6. The delivery mechanism of claim 1, wherein said odor-reducing quinone compound is obtained from a dye selected from the group consisting of Acid Blue 25, Acid Green 41, Acid Blue 45, Mordant Violet 5, Acid Blue 129, Acid Green 25, D&C Green No. 5, Acid Green 27, and combinations thereof.

7. The delivery mechanism of claim 1, wherein said high-surface area particles have an average size of less than about 100 nanometers, a surface area of from about 50 to about 1000 square meters per gram, and a pore volume of less than about 0.5 milliliters per gram.

8. The delivery mechanism of claim 7, wherein said high-surface area particles comprise alumina, silica, or combinations thereof.

9. The delivery mechanism of claim 7, wherein said high-surface area particles have a positive zeta potential.

10. A carrier vehicle for an odor control agent comprising: a porous cellulose-based substrate having a high-surface granule with at least about 200 m²/gram, which has free-flowing characteristics, and a quinone compound either adhered to or interfolded by said cellulose-based substrate.

11. The carrier vehicle according to claim 10, wherein said cellulose-based carrier substrate has a mean cross-sectional size ranging from about 150 μm to about 2500 μm.

12. The carrier vehicle according to claim 10, wherein said porous cellulose-based carrier substrate includes a synthetic polymer component.

13. The carrier vehicle according to claim 10, wherein said quinone compound is an anthraquinone having the following structure: 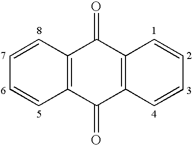
wherein the numbers 1 through 8 refer to optional substitution positions for functional groups.

14. The carrier vehicle according to claim 10, wherein each of said quinone compounds reacts with a molecule of a gaseous compound, an odorous compound, or combinations thereof.

15. The carrier vehicle according to claim 14, wherein said odorous compound comprises: a compound selected from the group consisting of sulfur-containing compounds, nitrogen-containing compounds, oxygen-containing compounds, and combinations thereof.

16. The carrier vehicle according to claim 10, wherein a number of said cellulose-based carrier substrates is free-flowing and do not exhibit a tendency to agglomerate to each other.

17. The carrier vehicle according to claim 10, wherein said carrier substrate has a surface area of at least about 200 m²/gram.

18. The carrier vehicle according to claim 10, wherein said cellulose-based carrier substrate has an average diameter of about 200-700 μm.

19. The carrier vehicle according to claim 10, wherein said cellulose-based carrier substrates exhibit free-flowing, powder-like characteristics, and have an average powder density of 30 to 600 g/l according to DIN 53,468.

20. The carrier vehicle according claim 19, wherein the granules have an average powder density according to DIN 53,468 amounting to 100 to 300 g/l.±0.15%.

21. The carrier vehicle granule according to claims 10, wherein cellulose fibers are present at least partially in the form of granules are partially also present in the form of fibers.

22. The carrier vehicle according to claim 10, wherein said cellulose fibers have an average fiber length of about 100 μm to about 600 μm.

23. The carrier vehicle according to claim 22, wherein said cellulose fibers have an average fiber length of about 300 μm.

24. The carrier vehicle according to claim 10, wherein said cellulose fibers have an average fiber thickness of about 10 μm to about 50 μm.

25. The carrier vehicle according to claim 24, wherein said cellulose fibers have an average fiber thickness of about 20 μm.

26. The carrier vehicle according to claim 10, wherein said cellulose fibers used to produce the granules are fine fibers, preferably fibers with an average fiber length of about 100 to 600 μm and an average fiber thickness of about 10 to 50 μm.

27. The carrier vehicle according to claim 10, wherein said odor control agent is encapsulated within said carrier substrate.

28. An article of manufacture comprising an odor control granule having a porous cellulose-based carrier substrate having a high-surface area of at least about 200 m²/gram, quinone compounds either adhered to or encapsulated within said cellulose-based substrate, wherein each quinone compound reacts with molecule of a gaseous compound, an odorous compound, or combinations thereof.

29. The article of manufacture according to claim 28, wherein said article is a personal care or absorbent article.

30. The article of manufacturer according to claim 28, wherein said article is a feminine hygiene product, a diaper, an adult incontinence product, or a protective garment.

31. The article of manufacturer according to claim 28, wherein said article is an air-freshener medium or package, air-filter medium.

32. The article of manufacture according to claim 28, wherein said article is nonwoven fabric constructed medium having said cellulose-based carrier substrates in interstial spaces of said nonwoven fabric.

33. An article of manufacturer comprising a cellulose-based granule or particle substrate with an odor control agent having an odor control composition including an odor-reducing quinone compound either adhered to a surface or interfolded among said cellulose-based granule.

34. The article of manufacture according to claim 33, wherein said article is an absorbent article comprising at least one liquid transmissive layer and a liquid absorbent core, wherein said liquid-transmissive layer, said liquid-absorbent core, or combinations thereof.

35. The article of manufacture according to claim 33, wherein said odor-reducing quinone compound is an anthraquinone having the following structure: 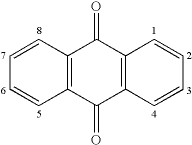
wherein the numbers 1 through 8 refer to optional substitution positions for functional groups.

36. The article of manufacture according to claim 35, wherein positions 5 through 8 of said anthraquinone are unsubstituted with functional groups.

37. The article of manufacture according to claim 33, wherein said odor-reducing quinone compound is obtained from a dye selected from the group consisting of Acid Blue 25, Acid Green 41, Acid Blue 45, Mordant Violet 5, Acid Blue 129, Acid Green 25, D&C Green No. 5, Acid Green 27, and combinations thereof.

38. The article of manufacture according to claim 33, wherein said odor control composition further comprises high-surface area particles having an average size of less than about 100 nanometers, a surface area of from about 50 to about 1000 square meters per gram, and a pore volume of less than about 0.5 milliliters per gram.

39. The article of manufacture according to claim 38, wherein said high-surface area particles have a positive zeta potential.

40. A method for delivering an odor control agent, said method comprising: selecting from the group consisting of quinone dyes or a quinone compound that is capable of reducing odor; forming an odor control composition that comprises said quinone compound; and adhering to a surface or interfolding said composition among a cellulose-based granule, and exposing said odor control composition to an odorous compound.

41. The method of claim 40, wherein said odor control composition has a dry solids add-on level of from about 0.1 wt. % to about 20 wt. %.

42. The method of claim 40, wherein said odor control composition has a dry solids add-on level of from about 0.5 wt. % to about 10 wt. %.

43. The method of claim 40, wherein said odor control composition is a powder.

44. The method of claim 40, wherein said odor control composition is an aqueous solution.

45. The method of claim 40, wherein said quinone compound is an anthraquinone having the following structure: 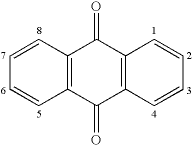
wherein the numbers 1 through 8 refer to optional substitution positions for functional groups.

46. The method of claim 45, wherein positions 5 through 8 of said anthraquinone are unsubstituted with functional groups.

47. The method of claim 40, wherein said quinone compound is selected from the group consisting of Acid Blue 25, Acid Green 41, Acid Blue 45, Mordant Violet 5, Acid Blue 129, Acid Green 25, D&C Green No. 5, Acid Green 27, and combinations thereof.

48. The method according to claim 40, wherein said cellulose-based granule has a surface area of at least about 200 m²/gram.

49. The method according to claim 40, wherein said cellulose-based granule exhibit free-flowing, powder-like characteristics, with an average powder density of 30 to 600 g/l according to DIN 53,468.
